(19) United States
(12) Patent Application Publication
(10) Pub. No.: US 2006/0161212 A1
(43) Pub. Date: Jul. 20, 2006

(54) METHOD AND DEVICE FOR DISPLAYING REMAINING SERVICEABLE LIFE OF AN ANALYTICAL DEVICE

(76) Inventors: Juergen Rasch-Menges, Schwetzingen (DE); Karl Werner, Wiesloch (DE); Frederic Wehowski, Hockenheim (DE)

Correspondence Address: Roche Diagnostics Corporation, Inc., 9115 Hague Road, PO Box 50457, Indianapolis, IN, 46250-0457, US

(21) Appl. No.: 11/276,134

(22) Filed: Feb. 15, 2006

Related U.S. Application Data
(63) Continuation of application No. PCT/EP04/08636, filed on Aug. 2, 2004.

(30) Foreign Application Priority Data
Aug. 16, 2003 (DE) 103 37 679.8

(51) Int. Cl. A61N 1/378

(52) U.S. Cl. 607/29

(57) ABSTRACT

The invention generally related to a method and device for displaying the remaining serviceable life of a battery-operated analytical device for analysis of a medically-relevant component of a body fluid, in particular of a blood glucose measuring device.

### REFERENCE TO RELATED APPLICATIONS

The present application is a continuation of PCT Patent Application No. PCT/EP2004/008636, filed Aug. 2, 2004 which claims priority to German Patent Application No. 103 37 679.8, filed Aug. 16, 2003, which are hereby incorporated by reference in their entirety.

### TECHNICAL FIELD

The invention generally concerns a method and a device for displaying the remaining serviceable life of a battery-operated analytical device.

### BACKGROUND

For qualitative and quantitative analysis of components of a liquid sample, in particular of a body fluid from humans or animals, test methods working with test elements are being used extensively. Typically these test elements contain reagents. In order to perform a reaction, the test element is contacted with the sample and the reaction between sample and reagent leads to a change in the test element that is characteristic of the analysis. The change in this characteristic of the sample is then analyzed using a suitable analytical device. Usually, the analytical device is suitable for analyzing a specific type of test elements made by a specific manufacturer. The test elements and the analytical device are mutually adapted components and, in combination, are called analytical system.

Different types of test elements are known which differ from each other by their measuring principle and the reagents used as well as by their set-up.

With regard to the measuring principle, colorimetric analytical systems are particularly common. In these systems, reaction of the sample with the reagents contained in the test element leads to a color changed that can be measured visually or by means of a photometric measuring facility. Aside from this, electrochemical analytical systems have gained great significance, in which the reaction of the sample with the reagents of the test element leads to an electrically detectable change (of an electrical voltage or an electrical current) that is measured with appropriate measuring electronics.

Often regular monitoring of certain analytic blood values is required. This applies in particular to diabetics who should monitor their blood sugar levels frequently in order to keep these levels within certain nominal limits by suitably adapting their insulin injections to the strongly varying needs.

Blood withdrawal systems should be easy to operate, have a compact, slim design, and be easy and cheap to manufacture. These practical requirements have lead and are leading to the development of blood analysis devices, which aim to satisfy these, to some extent contradictory, requirements to the extent possible.

Especially in the area of so-called “home monitoring”, i.e. where medical laymen perform simple blood analyses, and, in particular, in the periodical obtainment of blood several times daily by diabetics for control of their blood glucose concentration, it is important to have available an informative and reliable determination and display of the remaining serviceable life of the analytical device.

Blood glucose devices according to the prior art contain at least one battery, in particular round cells, for their energy supply. The batteries are placed in the devices in the course of their production. In order to provide for a basic storage period of the batteries until the first use of the device, a plastic adhesive is taped over the batteries with an insulating effect. This largely prevents discharging the battery prior to the first use of the device. The adhesive tape needs to be removed when the device is used the first time. This requires that the battery compartment is opened.

On a display of the device, a single symbol or icon is used to indicate the status of the batteries. In this context, the status of the batteries is determined from the difference between a nominal value of the battery charge and a current value of the battery charge. A user of the device is alerted to the need for battery replacement by means of the symbol. Accordingly, there is no exact display of the remaining serviceable life. Rather, only the approaching end of the battery life is indicated. Overall, this known battery replacement display according to the prior art is inexact and not truly reliable.

A disadvantage of the procedure according to the prior art is that it is not possible to activate a system clock of the device already during the production of the device because the voltage supply of the device occurs only once the user removes the adhesive tape. The first use of the device occurs after various lengths of time. Many users do not activate the system clock at all or incorrectly at the time of first use i.e. they do not set the system clock to the current time. As a result, measuring data in a measuring data memory of the device cannot be assigned to their measuring time. Reliable long-term monitoring is impossible in this case.

Resulting from differences in their history, e.g. different storage times of the devices after their production and the variation in the quality of the batteries used, in particular depending on the battery type and manufacturer, the reliability of the battery replacement display according to the prior art is poor, since the discharge characteristics of the batteries are unknown. A reliable remaining serviceable life display is not present. The battery status display mode described above contains too little information for this purpose, it is too one-dimensional.

Moreover, having to remove the adhesive tape or replace a battery, is associated with inconvenience and effort for the user of the device. The battery compartment must be opened, the correct type of battery must be procured, and the batteries must be inserted correctly. For these purposes, it may need to be necessary to consult the operating manual of the device.

Moreover, to have a reliable remaining serviceable life display indicating reliably to a user how many more measurements can be carried out with the set of batteries present in the analytical device, can be highly safety-relevant. For example diabetics depend on monitoring their blood glucose levels periodically and, if necessary, make appropriate corrections by administering medications. For these individuals, it is particularly important to be able to judge beforehand whether or not the blood glucose measuring device they take along, for example when traveling, will be available for a sufficient number of additional measurements. If the analytical device becomes inoperable unexpectedly, for example because a battery replacement display that is present according to the prior art is incapable of indicating a need for battery replacement sufficiently long ahead of time and a replacement battery is not rapidly available, even life-threatening conditions can occur due to the inability to carry out a blood glucose measurement.

Therefore, there is a need for displaying a reliable remaining serviceable life to allow a user of an analytical device to prepare for battery replacement in due time.

### SUMMARY

The invention is based on the object to provide a method for displaying the remaining serviceable life of battery-operated analytical devices and a corresponding analytical device that is set-up for carrying out the method, which overcome the disadvantages of the prior art and provide for an informative and reliable determination and display of the remaining serviceable life.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a first mode of displaying the remaining serviceable life of an analytical device according to the invention; and

FIG. 2 shows a second mode of displaying the remaining serviceable life of an analytical device according to the invention.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention.

In order that the invention may be more readily understood, reference is made to the following examples, which are intended to illustrate the invention, but not limit the scope thereof.

### DETAILED DESCRIPTION

The following description of the preferred embodiment is merely exemplary in nature and is in no way intended to limit the invention or its application or uses.

A display screen of an analytical device (not shown) is generally shown and represented by reference numeral 30. The display 30 provides for all displays needed for the functioning of the analytical device. For example, the display 30 may include information such as the current time, time of last measurement of the blood glucose etc.

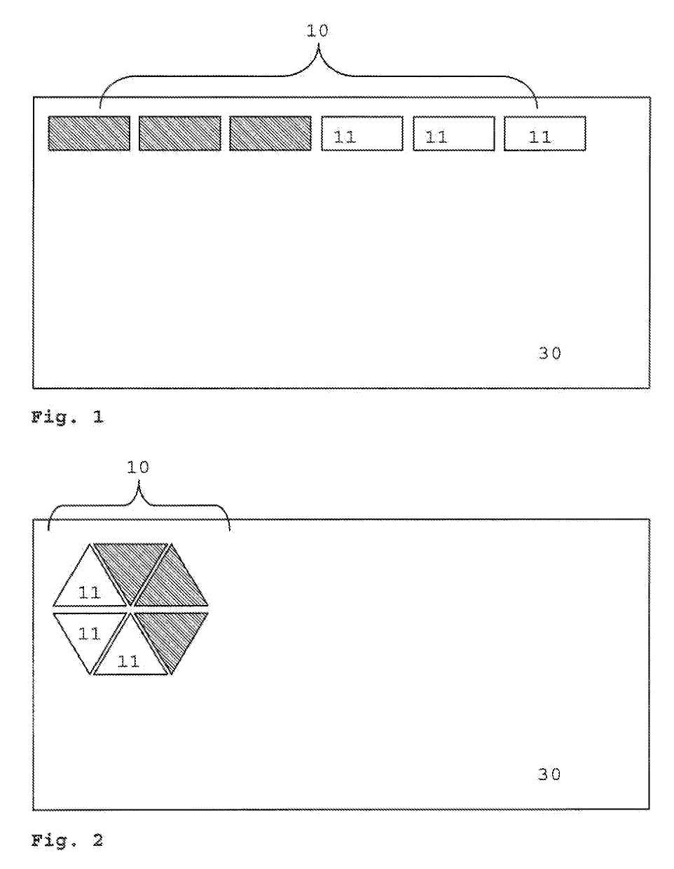

A part of the display 30 contains information regarding the remaining serviceable life of the analytical device. In other words, this displays the information on how many measurements are possible with the currently inserted battery. FIG. 1 shows a first mode of displaying the remaining serviceable life of an analytical device. For displaying the calculated remaining serviceable life, a display having multiple segments is shown and represented by reference numeral 10. Such displays are well known in the art and are commonly used in fuel indicators in motor vehicles.

These segments 10 indicate the degree of discharge of the batteries. In order to indicate the remaining life, the segments change their color or are made colorless according to the degree of discharge of the batteries, i.e. according to the calculated remaining serviceable life of the analytical device. The number of colored segments conveys to the user a prognosis of the remaining serviceable life. Each segment symbolizes, e.g., that another 50 or 300 measurements can be carried out with the analytical device.

As way of an example, FIG. 1 shows three colorless segments 11. The other three segments are colored, e.g. in a colored or black hatching. FIG. 1 shows a linear arrangement of six rectangular segments 10. FIG. 2 shows a modified arrangement of six triangular segments 10. However, the number of measurements that can be carried out still can also be displayed in a different fashion, e.g. as a number.

In order to calculate the remaining serviceable life of the analytical device, the device comprises an slot or a place for inserting a battery in the analytical device (not shown). Depending on the analytical device, the number of batteries requires to operate the analytical device may be one (1) or more than one.

The analytical device also comprises a system clock, a time measuring means for determining a battery service time that has clasped between a time of battery voltage supply and the current time. The battery voltage supply time is determined the time at which the analytical device was supplied with a battery voltage by the insertion of the battery. For the analytical device to calculate the remaining serviceable life, the analytical device also includes a calculating means for calculating a remaining number of measurements that can be carried out with the analytical device with the inserted battery by means of the battery service time and one or more further parameters of the analytical device and/or the battery inserted in the analytical device.

Accordingly, the time period between the time of insertion of the battery (time of battery voltage supply) in the analytical device and the current system time of the system clock is determined. This time period is then used in conjunction with other parameters to calculate the remaining serviceable life of the analytical device. The calculated remaining serviceable life in turn, is used to calculate the remaining number of measurements i.e. the number of measurements that can still be carried out with the battery currently inserted The latter is feasible since the energy consumption of the device for a single measurement is known or easy to determine, and is stored in the analytical device.

One of the parameters that can be taken into account for calculating the remaining serviceable life is to take into account the number of measurements carried out with the battery inserted in the analytical device. This allows the quality of the remaining serviceable life prediction to be improved.

Another parameter that can be taken into account in the calculation of the remaining number is at least one electrical parameter of the inserted battery. Battery parameters can be, for example, one or more of the following general parameters that are characteristic of the inserted type of battery: manufacturer, type of battery, maximal electrical charge stored in the battery, voltage-charge characteristics, discharge characteristics, self-discharge over time, temperature dependence. Alternatively or in addition, the following specific parameters of the battery can be taken into account: current terminal voltage, added-up value of charge drawn or current drawn, temperature, history (i.e. data concerning the aforementioned parameters that are stored in the analytical device), rest current.

It is also possible in some cases that the time of battery voltage supply is the insertion of the battery during the manufacture of the analytical device. In such situations, the system clock is activated from the time of battery voltage supply. Analytical devices, in particular blood glucose measuring devices, that are provided with batteries inserted at the time of production i.e. that are supplied with battery voltage from the time of their manufacture, and whose batteries are optimally selected for a predetermined number of measurements and matching serviceable life, allow the system clock, i.e. a built-in clock, to be activated and set correctly at the time of the manufacture. Accordingly, the time of the system clock is then set correctly.

Based on the aforementioned, a reliable value for the remaining serviceable life of the blood glucose measuring device is calculated from the time elapsed since manufacture, the number of measurements already carried out with the blood glucose device, and parameters of the batteries currently inserted in the device, and made available to the user of the device in a display suited for this purpose.

As shown in the drawings, the display of the remaining serviceable life is preferably implemented by means of a number of segments 10 representing an initial number of measurements. The remaining number is displayed by a number of segments that is proportional to the remaining number being displayed in a display mode different from the other segments that represent the initial number of measurements.

Preferably, the number of segments that is proportional to the remaining number is displayed by the segments in excess of this number of segments being displayed in a discolored fashion. The initial number of measurements can, for example, be a predetermined number of measurements that can be expected for an analytical device of a certain type with certain batteries that is set-up for carrying out the method according to the invention. As discussed above, the number of colored segments, for example, can then represent the number of measurements that are possible without replacing the current battery.

The remaining serviceable life display according to the invention implements a timely and very clear feedback mechanism for the remaining serviceable life of the analytical device.

The present invention can be applied both in analytical devices with user-replaceable batteries and in analytical devices whose batteries can be replaced only at that factory or by the customer service. In the former case, the effort involved in obtaining a reliable remaining serviceable life display is higher, though.

In the context of the present invention, “battery” is taken to mean any source of energy that can be inserted in or built into an analytical device and allows the analytical device to be operated independent of mains voltage. This includes not only disposable batteries, but also rechargeable batteries, so-called storage batteries. Considering their low self-discharge rate, round cells or RAM cells are preferred.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

Having described the invention in detail and by reference to specific embodiments thereof, it will be apparent that modification and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A Method for displaying the remaining serviceable life of a battery-operated analytical device for analysis of a medically-relevant component of a body fluid, the method comprising:
determining the battery service time that has elapsed between a time of battery voltage supply at which the analytical device was supplied with a battery voltage by inserting a battery, and the current time, whereby the battery service time is determined by means of a system clock of the analytical device;
calculating a remaining number of measurements that can be carried out with the analytical device with the inserted battery by means of the battery service time and one or more further parameters of the battery-operated analytical device and/or its battery; and
displaying the remaining serviceable life of the analytical by means of the calculated remaining number by means of a remaining serviceable life display of the analytical device displaying the remaining serviceable life or the remaining number.

2. The method according to claim 1, wherein the number of measurements carried out since the time of battery voltage supply with the battery inserted in the analytical device is taken into account as a parameter in the calculation of the remaining number.

3. The method according to claim 1, wherein least one electrical parameter of the inserted battery is taken into account as a parameter in the calculation of the remaining number.

4. The method according to claim 3, the general parameters that are characteristic of the inserted type of battery are taken into account as electrical parameter of the battery are selected from a group consisting of manufacturer, type of battery, maximal electrical charge stored in the battery, voltage-charge characteristics, discharge characteristics, self-discharge over time or temperature dependence.

5. The method according to claim 3, wherein one or more of the following specific parameters are taken into account as electrical parameter of the battery are selected from a group consisting of current terminal voltage, added-up value of charge drawn or current drawn, temperature, history, or rest current.

6. The method according to claim 1, wherein the time of battery voltage supply is the insertion of the battery during the manufacture of the analytical device, whereby the system clock is activated from the time of battery voltage supply.

7. The method according claim 1, wherein the display of the remaining serviceable life, a number of segments is used to display an initial number of measurements and the remaining number is displayed by a number of segments that is proportional to the remaining number being displayed in a display mode different from the other segments that represent the initial number of measurements.

8. A batter-operated analytical device for analysis of a medically-relevant component of a body fluid, the device comprising:
a system clock;
a time measuring means for determining a battery service time that has elapsed between a time of battery voltage supply at which the analytical device was supplied with a battery voltage by inserting a battery, and the current time, whereby the time measuring means determines the battery service time by means of the system clock;
a calculating means for calculating a remaining number of measurements that can still be carried out with the analytical device with the inserted battery by means of the battery service time and one or more further parameters of the batter-operated analytical device and/or its battery; and
a display for displaying the remaining serviceable life of the device by means of the calculated remaining number by means of a remaining serviceable life display of the analytical device displaying the remaining serviceable life or the remaining number.

9. The device according to claim 8, wherein the display comprises a number of segments for displaying an initial number of measurements and the remaining number can be displayed by a number of segments that is proportional to the remaining number being displayed in a different display mode from the other segments that represent the initial number of measurements.
